(19) United States
(12) Patent Application Publication
(10) Pub. No.: US 2005/0008667 A1
(43) Pub. Date: Jan. 13, 2005

(54) Cosmetic compositions for making up and/or caring for skin

(75) Inventors: Anne Liechty, Paris (FR); Euriel Clavel, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/853,785

(22) Filed: May 26, 2004

Related U.S. Application Data
(60) Provisional application No. 60/485,393, filed on Jul. 9, 2003.

(30) Foreign Application Priority Data
May 28, 2003 (FR) 03 06490

(51) Int. Cl. A61K 7/00

(52) U.S. Cl. 424/401

(57) ABSTRACT

The present invention relates to a cosmetic composition for caring for and/or making up the skin and/or the lips, comprising at least one liquid fatty phase, the said fatty phase containing at least one film-forming polymer and silicate particles.

### CROSS-REFERENCE TO RELATED APPLICATIONS

This application claims the benefit of French Application No. 03 06490 filed on May 28, 2003 and U.S. Provisional Application No. 60/485,393 filed on Jul. 9, 2003, the entire disclosures of which are incorporated by reference herein.

### BACKGROUND OF THE INVENTION

The present invention relates to cosmetic compositions for making up and/or caring for the skin, especially the face.

Compositions may especially be in the form of products cast in stick or dish form, for instance lipsticks or lip balms, cast foundations, concealer products, eyeshadows or makeup rouges, in more or less fluid paste or cream form, for instance fluid foundations or lipsticks, eyeliners, antisun compositions, skin-coloring compositions or body makeup compositions.

Makeup or care products for the human skin or lips, for instance foundations or lipsticks, generally contain a fatty phase based on oil(s) and/or wax(es), pigments and/or fillers and optionally additives, for instance cosmetic or dermatological active agents.

More particularly, compositions under consideration according to exemplary embodiments of the invention can constitute makeup products, for example for the face, in which the long-term staying power of the matt effect of the makeup is prolonged, i.e., the visual degradation of which over time is significantly reduced.

By definition, a matting product is a product that prevents the skin from shining and that unifies the complexion.

It is already known practice to use compositions with a matting effect. However, these compositions are not entirely satisfactory. They do not generally make it possible to provide a sustained matt effect over time and their application needs to be freshened regularly, especially beyond four hours of exposure, in order to ensure the expected effect.

There is thus a need for cosmetic compositions that can give a matt makeup effect with good staying power over time.

### SUMMARY OF THE INVENTION

The inventors have found, unexpectedly, that it is possible to overcome the above mentioned drawbacks by incorporating, into a fatty phase of a cosmetic composition, specific silicate particles in combination with at least one film-forming compound.

Consequently, in various exemplary embodiments, the present invention relates to cosmetic compositions for caring for and/or making up the skin and/or the lips, comprising at least one liquid fatty phase, the fatty phase containing at least one film-forming polymer and amorphous silicate particles.

In various exemplary embodiments, the invention includes cosmetic care and/or makeup processes comprising at least one step of application of a composition according to the invention to the skin and/or the lips.

In various exemplary embodiments, the invention is also directed towards use of silicate particles, especially in an amorphous form, according to the invention in matting cosmetic compositions, to obtain a matt effect that is sustained over time.

### DETAILED DESCRIPTION OF EXEMPLARY EMBODIMENTS

Silicate

Silicates according to the invention may especially have a specific surface area, measured according to the BET method, of greater than or equal to 100 m²/g, especially 200 m²/g and in particular 250 m²/g.

“BET specific surface area” is determined according to the BET (Brunauer-Emmet-Teller) method described in The Journal of the American Chemical Society, Vol. 60, page 309, February 1938 and corresponding to international standard ISO 5794/1 (appendix D). The BET specific surface area corresponds to the total specific surface area (thus including micropores) of a silicate under consideration.

Silicates according to exemplary embodiments of the invention are in the form of particles whose size may vary significantly. This size is generally adjusted with respect to cosmetic formulations under consideration.

In particular, more than 90%, especially more than 95% and in particular more than 98% of silicate particles may have a number-average size of less than or equal to 100 μm, especially less than or equal to 75 μm and in particular less than or equal to 50 μm, or even less than or equal to 30 μm.

Silicates may be present in a crystalline or, preferably, amorphous form. They are distinguished in this respect from silicates of talc type, which are naturally lamellar. Amorphous silicates according to exemplary embodiments of the invention are also generally porous.

In various exemplary embodiments, silicates are mixed silicates.

As used herein, “mixed silicate” refers to silicates of natural or synthetic origin comprising at least two cations belonging to at least two different groups of the Periodic Table and more particularly chosen from the following groups:

alkali metals, for instance sodium, lithium or potassium,

alkaline-earth metals, for instance beryllium, magnesium or calcium,

transition metals, and

aluminium.

According to one exemplary embodiment, mixed silicates can comprise as cations at least one aluminium cation, in particular combined with at least one cation of an alkaline-earth metal and especially of magnesium.

As non-limiting illustrations of amorphous mixed silicates that are suitable for the invention, mention may be made especially of magnesium aluminium silicate, especially the product sold under the name “Neusilin grade UFL2” by the company Fuji Chemical.

Silicate particles may be present in exemplary cosmetic compositions according to the invention in amounts ranging from 0.1% to 20% by weight, especially from 0.5% to 15% by weight and in particular from 1% to 10% by weight relative to the total weight of a composition.

Film-Forming Polymer

Exemplary compositions according to the invention can comprise at least one film-forming polymer, which may be chosen from liposoluble film-forming polymers, lipodispersible film-forming polymers, and blends thereof.

Liposoluble Polymer

Liposoluble polymers may be of any chemical nature and especially include:

(a) liposoluble, amorphous homopolymers and copolymers of olefins, of cycloolefins, of butadiene, of isoprene, of styrene, of vinyl ethers, esters or amides, or of (meth)acrylic acid esters or amides comprising a linear, branched or cyclic C₄-C₅₀ alkyl group. Liposoluble homopolymers and copolymers may especially be chosen from those obtained from monomers chosen from the group consisting of isooctyl(meth)acrylate, isononyl(meth)acrylate, 2-ethylhexyl(meth)acrylate, lauryl(meth)acrylate, isopentyl(meth)-acrylate, n-butyl(meth)acrylate, isobutyl(meth)acrylate, tert-butyl (meth)acrylate, tridecyl (meth)acrylate and stearyl (meth)acrylate, or mixtures thereof. Examples that will be mentioned include the alkyl acrylate/cycloalkyl acrylate copolymer sold by Phoenix Chem under the name Giovarez AC-5099 ML.

Liposoluble film-forming polymers that may also be mentioned include vinylpyrrolidone (VP) copolymers and especially copolymers of vinylpyrrolidone and of a C₂-C₄₀ and in particular C₃ to C₂₀ alkene. As examples of VP copolymers that may be used in the invention, mention may be made of copolymers of VP/vinyl acetate, VP/ethyl methacrylate, VP/ethyl methacrylate/methacrylic acid, VP/eicosene, VP/hexadecene, VP/triacontene, VP/styrene, VP/acrylic acid/lauryl methacrylate and butylated polyvinylpyrrolidone (PVP).

Particular liposoluble copolymers that may also be mentioned include:

(i) acrylic-silicone grafted polymers containing a silicone skeleton and acrylic grafts or containing an acrylic skeleton and silicone grafts, such as the products sold under the names “SA 70.5”, “SA 70” and “VS 80” by 3M and described especially in U.S. Pat. No. 5,725,882, U.S. Pat. No. 5,209,924, U.S. Pat. No. 4,972,037, U.S. Pat. No. 4,981,903, U.S. Pat. No. 4,981,902, U.S. Pat. No. 5,468,477, U.S. Pat. No. 5,219,560, EP 0 388 582, U.S. Pat. No. 5,032,460 and WO 93/23009,

(ii) liposoluble polymers belonging to one of the classes described above and bearing fluoro groups, in particular those described in U.S. Pat. No. 5,948,393 and the alkyl (meth)acrylate/perfluoroalkyl (meth)acrylate copolymers described in EP 0 815 836 and U.S. Pat. No. 5,849,318,

(iii) polymers or copolymers resulting from the polymerization or copolymerization of an ethylenic monomer, comprising one or more ethylenic bonds, which are preferably conjugated (or diene). This or these agent(s) is (are) in particular vinyl, acrylic or methacrylic copolymers, which may be in block form and especially of diblock or triblock type, or even of multiblock or starburst type.

Ethylenic film-forming polymers may especially comprise a styrene (S) block, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks.

In particular, film-forming polymers used in various exemplary embodiments may be copolymers comprising at least one styrene block. Most particularly, a triblock copolymer may be used and in particular those of the polystyrene/polyisoprene or polystyrene/polybutadiene type, such as those sold under the name “Luvitol HSB” by BASF, and those of the polystyrene/copoly(ethylene-propylene) type or alternatively of the polystyrene/copoly(ethylene-butylene) type, such as those sold under the brand name “Kraton” by Shell Chemical Co. or Gelled Permethyl 99A by Penreco. Styrene-methacrylate copolymers may also be used.

As film-forming polymers that may be used in exemplary compositions of the invention, examples that may also be mentioned include Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton G1750X (EP) multiarm, Kraton G1765X (EP) multiarm, Kraton D-1101 (SBS), Kraton D-1102 (SBS), Kraton D-1107 (SiS), Gelled Permethyl 99A-750, Gelled Permethyl 99A-753-58 (blend of triblock and of starburst block polymer), Gelled Permethyl 99A-753-59 (blend of triblock and of starburst block polymer), Versagel 5970 and Versagel 5960 from Penreco (blend of triblock and of starburst polymer in isododecane), and OS 129880, OS 129881 and OS 84383 from Lubrizol (styrene-methacrylate copolymer);

(b) amorphous liposoluble polycondensates, in particular those containing no groups that give hydrogen interaction, especially polyesters containing C₄-C₅₀ alkyl side chains or polyesters resulting from the condensation of fatty acid dimers, or alternatively polyesters comprising a silicone segment in block, graft or terminal group form, which are solid at room temperature, for example as defined in patent application FR-A-2831430;

(c) amorphous liposoluble polysaccharides comprising alkyl (ether or ester) side chains, in particular alkylcelluloses comprising a linear or branched, saturated or unsaturated C₁-C₈ alkyl radical such as ethylcellulose and propylcellulose.

In general, film-forming liposoluble polymers of exemplary embodiments of the invention may have a weight-average molecular weight ranging from 1000 to 500 000 and especially from 2000 to 250 000, and a glass transition temperature ranging from −100° C. to +300° C., especially from −50° C. to +100° C. and in particular from −10° C. to +90° C.

Lipodispersible Polymer

Lipodispersible polymers are generally present in the form of a stable dispersion of particles, which are generally spherical, in the liquid fatty phase. These dispersions may especially be in the form of polymer nanoparticles in dispersion. These nanoparticles may have a size ranging from 5 to 600 nm and especially ranging from 50 to 250 nm.

Polymers of the dispersed particles that may be used in exemplary compositions according to the invention may have a weight-average molecular weight ranging from about 2000 to 10 000 000.

Exemplary polymers may have a glass transition temperature ranging from −100° C. to +300° C., especially from −10° C. to +50° C. and more particularly less than or equal to about +40° C.

Polymers used in exemplary embodiments of the present invention in the form of particles dispersed in a fatty phase may be of any nature. It is thus possible to use a free-radical polymer, a polycondensate or even a polymer of natural origin, and blends thereof. Polymers may be chosen by a person skilled in the art on the basis of its properties.

Exemplary polymers include “film-forming” polymers, i.e., polymers capable of forming an isolable film, by themselves or in combination with a plasticizer.

Illustrations of film-forming polymers that may be mentioned include acrylic or vinyl free-radical homopolymers or copolymers, especially those with a glass transition temperature (Tg) of less than or equal to about +40° C. and in particular ranging from −10° C. to +30° C., and mixtures thereof.

The expression “free-radical polymer” refers to polymers obtained by polymerization of monomers containing unsaturation, especially ethylenic unsaturation, each monomer being capable of homopolymerizing (unlike polycondensates). Free-radical polymers may especially be vinyl polymers or copolymers, especially acrylic polymers.

Vinyl polymers may result from polymerization of ethylenically unsaturated monomers containing at least one acid group and/or esters of these acidic monomers and/or amides of these acids.

As monomers bearing an acidic group, it is possible to use α,β-ethylenic unsaturated carboxylic acids such as acrylic acid, methacrylic acid, crotonic acid, maleic acid or itaconic acid. (Meth)acrylic acid and crotonic acid are especially used, and more particularly (meth)acrylic acid.

Esters of acidic monomers are advantageously chosen from esters of (meth)acrylic acid (also known as (meth)acrylates), for instance alkyl (meth)acrylates, in particular of a C₁-C₂₀ and more particularly a C₁-C₈ alkyl, aryl (meth)acrylates, in particular of a C₆-C₁₀ aryl, and hydroxyalkyl (meth)acrylates, in particular of a C₂-C₆ hydroxyalkyl.

Alkyl(meth)acrylates which may be mentioned include methyl, ethyl, butyl, isobutyl, 2-ethylhexyl and lauryl (meth)acrylate.

Hydroxyalkyl(meth)acrylates which may be mentioned include hydroxyethyl(meth)acrylate and 2-hydroxypropyl(meth)acrylate.

Aryl (meth)acrylates which may be mentioned include benzyl and phenyl acrylate.

(Meth)acrylic acid esters that are particularly preferred are the alkyl (meth)acrylates.

Free-radical polymers that are especially used are copolymers of (meth)acrylic acid and of alkyl(meth)acrylate, especially of a C₁-C₄ alkyl. More particularly, methyl acrylates may be used, optionally copolymerized with acrylic acid, such as copolymers of poly(methyl acrylate/acrylic acid) type.

Amides of acidic monomers which may be mentioned include (meth)acrylamides, and especially N-alkyl(meth)acrylamides, in particular of a C₂-C₁₂ alkyl, such as N-ethylacrylamide, N-t-butylacrylamide and N-octylacrylamide; N-di(C₁-C₄)alkyl(meth)acrylamides.

Vinyl polymers may also result from polymerization of ethylenically unsaturated monomers containing at least one amine group, in free form or partially or totally neutralized, or alternatively partially or totally quaternized. Such monomers may be, for example, dimethylaminoethyl(meth)acrylate, dimethylaminoethylmethacrylamide, vinylamine, vinylpyridine and diallyldimethylammonium chloride.

Vinyl polymers may also result from homopolymerization or copolymerization of at least one monomer chosen from vinyl esters and styrene monomers. In particular, these monomers may be polymerized with acidic monomers and/or esters thereof and/or amides thereof, such as those mentioned above.

Examples of vinyl esters which may be mentioned include vinyl acetate, vinyl propionate, vinyl neodecanoate, vinyl pivalate, vinyl benzoate and vinyl t-butylbenzoate.

Styrene monomers which may be mentioned include styrene and α-methylstyrene.

The list of monomers given is not limiting, and it is possible to use any monomer known to those skilled in the art which falls within the categories of acrylic and vinyl monomers (including monomers modified with a silicone chain).

As other vinyl monomers that may be used, mention may also be made of:

N-vinylpyrrolidone, vinylcaprolactam, vinyl-N-(C₁-C₆)alkylpyrroles, vinyloxazoles, vinylthiazoles, vinylpyrimidines and vinylimidazoles; and

olefins such as ethylene, propylene, butylene, isoprene and butadiene.

Vinyl polymers may be crosslinked with one or more difunctional monomers, especially comprising at least two ethylenic unsaturations, such as ethylene glycol dimethacrylate or diallyl phthalate.

In a non-limiting manner, exemplary polymers according to the invention may be chosen from the following polymers or copolymers: polyurethanes, polyurethane-acrylics, polyureas, polyurea-polyurethanes, polyester-polyurethanes, polyether-polyurethanes, polyesters, polyesteramides, fatty-chain polyesters, alkyds; acrylic and/or vinyl polymers or copolymers; acrylic-silicone copolymers; polyacrylamides; silicone polymers; fluoro polymers and mixtures thereof.

According to exemplary embodiments of the invention, lipodispersible polymers may be surface-stabilized with at least one liposoluble polymer, for instance those described in patent application EP 1 002 528, or those here-above described.

Film-forming polymer dispersions may be prepared as described in document EP-A-749 747 or in document EP 1 002 528. More specifically, polymerization is performed in dispersion, i.e., by precipitation of the polymer as it is formed, with protection of the formed particles with a stabilizer. Stabilizers are especially described in patent application EP 749 747.

In general, exemplary compositions according to the invention may comprise film-forming polymers in a solid content ranging from 0.1% to 25% by weight, especially from 1% to 20% by weight and in particular from 5% to 16% by weight, relative to the total weight of a composition.

Exemplary compositions according to the invention may comprise at least one auxiliary film-forming agent that promotes the formation of a film with the film-forming polymer. Such a film-forming agent may be chosen from any compound known to those skilled in the art as being capable of satisfying the desired function, and may be chosen especially from plasticizers and coalescers.

Coalescers or plasticizers that may be used in exemplary embodiments of the invention are especially those mentioned in document FR-A-2 782 917.

Liquid Fatty Phase

Exemplary liquid fatty phases may comprise one or more cosmetically or dermatologically acceptable and generally physiologically acceptable oils.

Thus, exemplary liquid fatty phases may comprise at least one oil, which may be chosen especially from carbon-based, hydrocarbon-based, fluoro-based and/or silicone-based oils of mineral, animal, plant or synthetic origin, alone or as a mixture, provided that they form a uniform, stable mixture and that they are compatible with an intended use.

The term “liquid fatty phase” refers to any non-aqueous medium that is liquid at room temperature (25° C.) and atmospheric pressure. A fatty phase may contain a volatile liquid fatty phase and/or a non-volatile fatty phase.

The term “volatile fatty phase” refers to any non-aqueous medium capable of evaporating from the skin or the lips in less than one hour. A volatile phase especially comprises oils with a vapour pressure, at room temperature and atmospheric pressure, ranging from 10⁻³ to 300 mmHg (0.13 Pa to 40 000 Pa).

Liquid fatty phases according to exemplary embodiments of the invention may comprise at least one compound chosen from hydrocarbon-based oils such as liquid paraffin or liquid petroleum jelly, mink oil, turtle oil, soybean oil, perhydrosqualene, sweet almond oil, beauty-leaf oil, palm oil, grapeseed oil, sesame seed oil, maize oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol, oleyl alcohol, linoleyl alcohol, linolenyl alcohol, isostearyl alcohol or octyldodecanol; silicone oils such as polydimethylsiloxanes (PDMS), which are optionally phenylated, such as phenyl trimethicones, or optionally substituted with aliphatic and/or aromatic groups, which are optionally fluorinated, or optionally substituted with functional groups such as hydroxyl, thiol and/or amine groups; polysiloxanes modified with fatty acids, fatty alcohols or polyoxyalkylenes, fluorosilicones and perfluoro oils.

Advantageously, one or more oils that are volatile at room temperature may be used.

They may be hydrocarbon-based oils or silicone oils and may optionally comprise alkyl or alkoxy groups, which are pendent or at the end of a silicone chain.

As volatile oils that may be used in the invention, mention may be made of linear or cyclic silicones containing from 2 to 7 silicon atoms, these silicones optionally comprising alkyl or alkoxy groups containing from 1 to 10 carbon atoms, especially octamethylcyclotetrasiloxane, decamethylcyclopentasiloxane, hexadecamethylcyclohexasiloxane heptamethylhexyltrisiloxane and heptamethyloctyltrisiloxane, and also C₈-C₁₆ isoparaffins such as the Isopar® and Permethyl® products, and especially isododecane.

Liquid fatty phase of exemplary compositions may represent from 5% to 97% by weight and especially from 20% to 85% by weight relative to the total weight of a composition. Non-volatile parts may represent from 0.1% to 80% by weight and especially from 1% to 50% by weight relative to the total weight of a composition.

Solid Fatty Substances

Fatty phases of exemplary compositions may also comprise at least one fatty substance that is pasty or solid at room temperature, chosen, for example, from waxes and gums, and mixtures thereof.

As used herein, the term “wax” means a lipophilic fatty compound that is solid at room temperature (25° C.), with a reversible solid/liquid change of state, having a melting point of greater than 30° C., which may be up to 200° C., a hardness of greater than 0.5 MPa, and an anisotropic crystal organization in the solid state. By bringing waxes to their melting points, it is possible to make them miscible with oils and to form a microscopically homogeneous mixture, but on returning the temperature of a mixture to room temperature, recrystallization of waxes in oils of a mixture is obtained.

Hardnesses of waxes are determined by measuring the compression force, measured at 20° C. using a texturometer sold under the name TA-XT2i by the company Rheo, equipped with a stainless-steel cylinder 2 mm in diameter travelling at a measuring speed of 0.1 mm/s and penetrating into the wax to a penetration depth of 0.3 mm. To perform a hardness measurement, waxes are melted at a temperature equal to a melting point of the waxes +20° C. Molten waxes are poured into a container 30 mm in diameter and 20 mm deep. Waxes are recrystallized at room temperature (25° C.) for 24 hours and are then stored for at least one hour at 20° C. before performing the hardness measurement. Values of hardnesses are measured compression forces divided by an area of the texturometer cylinder in contact with the waxes.

In various exemplary embodiments, waxes may be hydrocarbon-based waxes, silicone waxes and/or fluoro waxes and may optionally comprise ester or hydroxyl functions. They may especially be of natural or synthetic origin. Non-limiting illustrations of waxes that may especially be mentioned include optionally modified beeswax, carnauba wax, candelilla wax, ouricury wax, Japan wax, cork fibre wax or sugarcane wax, ceresin, paraffin wax, lignite wax, microcrystalline waxes, lanolin wax, montan wax, ozokerites, hydrogenated oils, for instance hydrogenated jojoba oil or ethylene copolymerization oil, waxes obtained by Fischer-Tropsch synthesis, fatty acid esters and glycerides that are solid at 45° C., silicone waxes, for instance alkyl, alkoxy and/or esters of poly(di)methylsiloxane that are solid at 45° C., containing from 10 to 45 carbon atoms, and certain fatty acids, for instance stearic acid, myristic acid or behenic acid, and mixtures thereof.

Exemplary compositions according to the invention may comprise at least one wax in a content ranging from 0.1% to 20% by weight, especially from 2% to 15% by weight and in particular from 3% to 12% by weight relative to the total weight of a composition.

Aqueous Phase

Exemplary compositions according to the invention may also comprise an aqueous phase containing water. Water may be a floral water such as cornflower water and/or a mineral water such as eau de Vittel, eau de Lucas or eau de La Roche Posay and/or a spring water.

Aqueous phases may also comprise solvents other than water, for instance primary alcohols such as ethanol and isopropanol, glycols such as glycerol, propylene glycol, butylene glycol, dipropylene glycol, diethylene glycol, glycol ethers, such as C₁-C₄ alkyl ethers of mono-, di- or tripropylene glycol, or mono-, di- or triethylene glycol, and mixtures thereof.

Aqueous phases may also comprise stabilizers, for example sodium chloride, magnesium dichloride and magnesium sulphate.

Aqueous phases may also comprise any water-soluble or water-dispersible compound that is compatible with an aqueous phase, such as gelling agents, film-forming polymers, thickeners and surfactants, and mixtures thereof.

Aqueous phases, especially water, may especially be present in the composition according to the invention in a content ranging from 2% to 75% by weight and in particular from 5% to 50% by weight relative to the total weight of the composition.

Compositions may be present in the form of an oil-in-water or water-in-oil emulsion. Compositions may also be present in anhydrous form.

When exemplary compositions according to the invention are in the form of emulsions, they may also comprise a surfactant or a mixture of surfactants whose HLB (hydrophilic/lipophilic balance) value is generally suited to the nature of the emulsion to be stabilized.

As surfactants that may be used in the invention, which are suitable for producing a W/O emulsion, mention may be made of those with an HLB value of less than 7 and especially fatty acid esters of polyols, for instance glyceryl or sorbitol mono-, di-, tri- or sesquioleates, and glyceryl or polyethylene glycol laurates; alkyl or alkoxy dimethicone copolyols containing an alkyl or alkoxy chain that is pendent or at the end of a silicone skeleton, containing, for example, from 6 to 22 carbon atoms. As surfactants that may be used in the invention to produce an O/W emulsion, mention may be made of those with an HLB value of greater than 7, for instance fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl) ethers and dimethicone copolyols. In general, any amphoteric ionic (cationic or anionic) surfactant and any nonionic surfactant, which are well known to those skilled in the art, may be used.

Surfactants may be present in exemplary compositions in amounts ranging from 0.3% to 10% by weight and especially from 1% to 5% by weight relative to the total weight of a composition.

Exemplary compositions according to the invention may also comprise at least one additional pulverulent compound, i.e., a compound other than the silicates considered according to the invention. Additional pulverulent compounds may be chosen from fillers, pulverulent dyestuffs such as pigments and nacres, and mixtures thereof.

The term “fillers” refers to white or colorless, mineral or synthetic, lamellar or non-lamellar particles.

Fillers may be chosen from talc, mica, silica, kaolin, poly-β-alanine powder, polyethylene powder, powders of lauroyllysine, starch, boron nitride, hollow polymer microspheres such as those of polyvinylidene chloride/acrylonitrile, such as Expancel® (Nobel Industrie), acrylic polymer particles, especially of acrylic acid copolymer, for instance Polytrap® (Dow Coming), polyurethane powders, silicone resin microbeads (for example Tospearls® from Toshiba), precipitated calcium carbonate, dicalcium phosphate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc stearate, magnesium stearate or lithium stearate, zinc laurate and magnesium myristate, and mixtures thereof.

Fillers may or may not be surface-treated, especially to make them lipophilic.

Fillers may be present in exemplary compositions in amounts ranging from 0.1% to 30% by weight and especially from 0.1% to 10% by weight relative to the total weight of a composition.

As used herein, the term “pigments” is intended to denote white or colorless, mineral or organic particles that are insoluble in the liquid organic phase, which are intended to color and/or opacify the composition.

Pigments may be mineral and/or organic pigments. Pigments that may be used include metal oxides, for instance iron oxides (especially yellow, red, brown or black iron oxides), titanium dioxides, cerium oxide, zirconium oxide and chromium oxide; manganese violet, ultramarine blue, Prussian blue, ferric blue, bismuth oxychloride, natural mother-of-pearl, mica coated with titanium or with bismuth oxychloride, colored nacreous pigments such as titanium mica with iron oxides, titanium mica especially with ferric blue or chromium oxide, titanium mica with an organic pigment of the above mentioned type and nacreous pigments based on bismuth oxychloride, and mixtures thereof.

Iron oxide or titanium dioxide pigments are used in particular.

Pigments may be treated, where appropriate, with a hydrophobic agent to make them compatible with a fatty phase of a composition.

Such hydrophobic-treated pigments are especially described in patent application EP-A-1 086 683.

As used herein, the term “nacres” refers to iridescent particles, produced especially by certain molluscs in their shell or else synthesized, which are insoluble in the medium of a composition.

Pulverulent dyestuffs may be present in a content ranging from 0.5% to 30% by weight, in particular ranging from 5% to 20% by weight and especially ranging from 6% to 15% by weight relative to the total weight of a composition.

Exemplary compositions may also comprise at least one lipophilic dye and/or at least one hydrophilic dye.

As used herein, the term “dye” refers to compounds, generally organic compounds, which are soluble in the fatty substances such as the oils or in an aqueous-alcoholic phase.

Among liposoluble dyes that may be used in exemplary embodiments of the invention, mention may be made of Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

Exemplary compositions according to the invention may contain one or more adjuvants that are common in cosmetics, such as hydrophilic or lipophilic gelling agents and/or thickeners; moisturizers; emollients; hydrophilic or lipophilic active agents; free-radical scavengers; sequestering agents; antioxidants; preserving agents; basifying or acidifying agents; fragrances; and mixtures thereof.

As active agents that may be used in exemplary compositions according to the invention, mention may be made, for example, of moisturizers such as protein hydrolysates and polyols, for instance glycerol, glycols, for instance polyethylene glycols, and sugar derivatives; natural extracts; antiinflammatory agents; procyannidol oligomers; vitamins such as vitamin A (retinol), vitamin E (tocopherol), vitamin C (ascorbic acid), vitamin B5 (panthenol), vitamin B3 (niacinamide), derivatives of these vitamins (especially esters) and mixtures thereof; urea; caffeine; salicylic acid and its derivatives; α-hydroxy acids such as lactic acid and glycolic acid and derivatives thereof; retinoids such as carotenoids and vitamin A derivatives; sunscreens; hydrocortisone; melatonin; algal, fungal, plant, yeast or bacterial extracts; enzymes; steroids; antibacterial active agents, for instance 2,4,4′-trichloro-2′-hydroxydiphenyl ether (or triclosan), 3,4,4′-trichlorocarbanilide (or triclocarban) and the acids indicated above, and especially salicylic acid and its derivatives; tensioning agents; and mixtures thereof.

Exemplary compositions according to the invention may also comprise at least one sunscreen (or UV-screening agent). Agents may be chosen from organic screening agents and physical sunblocks, and mixtures thereof.

As chemical sunscreens, exemplary compositions of the invention may comprise any UVA- and UVB-screening agents that may be used in cosmetics.

Needless to say, a person skilled in the art will take care to select optional adjuvant(s) added to the composition according to the invention and to adjust the amounts thereof such that the advantageous properties intrinsically associated with the composition in accordance with the invention are not, or are not substantially, adversely affected by the envisaged addition.

This invention is illustrated by the following Examples, which are merely for the purpose of illustration.

### EXAMPLE 1

### Preparation of a Dispersion of a Film-Forming Polymer

A dispersion of a non-crosslinked copolymer of methyl acrylate and of acrylic acid in an 85/15 ratio, in isododecane, is prepared according to the method of Example 7 of document EP-A-749 747. A dispersion of poly(methyl acrylate/acrylic acid) particles surface-stabilized in isododecane with a polystyrene/copoly(ethylene-propylene) diblock copolymer sold under the name Kraton G1701 (Shell), having a solids content of 22.6% by weight, a mean particle size of 175 nm (polydispersity: 0.05) and a Tg of 20° C., is thus obtained. This copolymer can form a film at room temperature (25° C.).

### EXAMPLE 2

A foundation in the form of a water-in-oil emulsion having the composition below is prepared:

Oily phase:

| | |
|---|---|
| Dispersion of film-forming polymer of Example 1 | 55.52 g |
| Isododecane gelled with Kraton (triblock and radial) | 4.48 g |
| sold under the trade name Versagel M5950 by the | |
| company Penreco | |
| Isododecane | 1.97 g |
| Blend of polydimethylsiloxane containing α-ω oxyethylene | 0.90 g |
| oxypropylene groups (DP: 100-58/42) and of | |
| cyclopenta-siloxane (85/15) sold under the trade name | |
| “Abil EM 97” by the company Goldschmidt | |
| Cyclopentadimethylsiloxane | 4.00 g |
| Nylon 12 powder sold under the trade name Orgasol 2002 | 3.00 g |
| Extra D Nat. Cos. by the company Atochem | |
| Hydrated magnesium aluminium silicate sold under | 4.00 g |
| the trade name Neusilin UFL2 by the company Fuji Chemical | |
| Mixture of disteardimonium hectorite, cyclopentasiloxane | 3.53 g |
| and ethanol (10/85/5) (Bentone Gel V5-5V from | |
| Elementis Specialities) | |
| Isostearic acid mono-di-glycerides esterified with | 0.30 g |
| succinic acid, non-stabilized, sold under the trade | |
| name “Inwitor 780 K” by the company Sasol | |
| Iron oxides coated with aluminium stearoylglutamate | 3.21 g |
| Titanium oxide coated with aluminium stearoylglutamate | 6.79 g |

Aqueous phase:

| Glycerol | 3.00 | g |
|---|---|---|
| Magnesium sulfate | 0.70 | g |
| Preserving agents | qs | |
| Water | qs 100.00 | g |

The emulsion is prepared by mixing together, at room temperature, the constituents of the oily phase and then adding the aqueous phase, prepared beforehand, with stirring.

The foundation thus obtained applies easily to the skin and has a good feeling of creaminess, and the makeup result obtained affords a soft deposit and the matt effect at the time of application is satisfactory and persists for well beyond four hours.

While this invention has been described in conjunction with the exemplary embodiments and examples outlined above, various alternatives, modifications, variations, improvements and/or substantial equivalents, whether known or that are or may be presently unforeseen, may become apparent to those having at least ordinary skill in the art. Accordingly, the exemplary embodiments of the invention, as set forth above, are intended to be illustrative, not limiting. Various changes may be made without departing from the spirit and scope of the invention. Therefore, the invention is intended to embrace all known or later developed alternatives, modifications, variations, improvements and/or substantial equivalents.

1. Cosmetic composition for caring for and/or making up the skin and/or the lips, comprising at least one liquid fatty phase, the said fatty phase containing at least one film-forming polymer and silicate particles.

2. Composition according to claim 1, characterized in that the silicate is a mixed silicate.

3. Composition according to claim 2, characterized in that the mixed silicate comprises at least two cations belonging to at least two different groups of the Periodic Table chosen from the group of alkali metals, the group of alkaline-earth metals, the group of transition metals and aluminium.

4. Composition according to either of claims 2 and 3, characterized in that the mixed silicate comprises at least one aluminium cation.

5. Composition according to claim 4, characterized in that the aluminium cation is combined with at least one cation from the group of alkaline-earth metals.

6. Composition according to any one of claims 2 to 5, characterized in that the mixed silicate is a magnesium aluminium silicate.

7. Composition according to any one of the preceding claims, characterized in that more than 90%, especially more than 95% and in particular more than 98% of the silicate particles have a number-average size of less than or equal to 100 μm, especially 75 μm and in particular 50 μm.

8. Composition according to any one of the preceding claims, characterized in that the silicate particles have a specific surface area, measured according to the BET method, of greater than or equal to 100 m²/g, especially 200 m²/g and in particular 250 m²/g.

9. Composition according to any one of the preceding claims, characterized in that the silicate is porous.

10. Composition according to any one of the preceding claims, characterized in that it comprises silicate particles in a content ranging from 0.1% to 20% by weight, especially from 0.5% to 15% by weight and in particular from 1% to 10% by weight, relative to the total weight of the composition.

11. Compostion according to any one of the preceding claims, characterized in that the film-forming polymer is chosen from liposoluble and lipodispersible film-forming polymers and mixtures thereof.

12. Composition according to claim 11, characterized in that the liposoluble film-forming polymer is chosen from liposoluble, amorphous homopolymers and copolymers of olefins, of cycloolefins, of butadiene, of isoprene, of styrene, of vinyl ethers, esters or amides, or of (meth)acrylic acid esters or amides comprising a linear, branched or cyclic C₄₋₅₀ alkyl group; amorphous liposoluble polycondensates, especially polyesters containing C₄₋₅₀ alkyl side chains or polyesters resulting from the condensation of fatty acid dimers; liposoluble amorphous polysaccharides comprising alkyl (ether or ester) side chains; vinylpyrrolidone (VP) copolymers; acrylic silicone grafted polymers containing an acrylic skeleton and silicone grafts or containing a silicone skeleton and acrylic grafts; and blends thereof.

13. Composition according to claim 11 or 12, characterized in that the film-forming polymer comprises at least one triblock copolymer and in particular those of the polystyrene/polyisoprene or polystyrene/polybutadiene type or alternatively of the polystyrene/copoly(ethylene-butylene) or polystyrene/copoly(ethylene-propylene) type.

14. Composition according to any one of claims 11 to 13, characterized in that the lipodispersible polymer is chosen from polyurethanes, polyurethane-acrylics, polyureas, polyurea-polyurethanes, polyester-polyurethanes, polyether-polyurethanes, polyesters, polyesteramides, fatty-chain polyesters, alkyds, acrylic and/or vinyl polymers or copolymers, acrylic-silicone copolymers, polyacrylamides, silicone polymers and fluoro polymers, and blends thereof.

15. Composition according to claim 14, characterized in that the lipodispersible polymer is chosen from acrylic or vinyl free-radical homopolymers and copolymers, especially having a glass transition temperature (Tg) of less than or equal to 40° C. and in particular ranging from −10 to 30° C., and blends thereof.

16. Composition according to claim 14 or 15, characterized in that the lipodispersible polymer is an acrylic polymer of poly(methyl acrylate/acrylic acid) type.

17. Composition according to any one of claims 14 to 16, characterized in that the said lipodispersible film-forming polymer is surface-stabilized with at least one liposoluble film-forming polymer, especially as defined according to either of claims 12 and 13.

18. Composition according to any one of the preceding claims, characterized in that the film-forming polymer is present in a solids content ranging from 0.1% to 25% by weight, especially from 1% to 20% by weight and in particular from 5% to 16% by weight relative to the total weight of the composition.

19. Composition according to any one of the preceding claims, characterized in that the fatty phase comprises at least one oil chosen from carbon-based, hydrocarbon-based, fluoro-based and/or silicone-based oils of mineral, animal, plant or synthetic origin, and mixtures thereof.

20. Composition according to any one of the preceding claims, characterized in that the liquid fatty phase comprises at least one compound chosen from liquid paraffin or liquid petroleum jelly, mink oil, turtle oil, soybean oil, perhydrosqualene, sweet almond oil, Calophyllum oil, palm oil, parleam oil, grapeseed oil, sesame seed oil, maize oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleic alcohol or linoleic, isostearyl alcohol or octyldodecanol; silicone oils such as polydimethylsiloxanes (PDMS), which are optionally phenylated, such as phenyl trimethicones, or optionally substituted with aliphatic and/or aromatic groups, or optionally substituted with functional groups such as hydroxyl, thiol and/or amine groups; polysiloxanes modified with fatty acids, fatty alcohols or polyoxyalkylenes, fluoro oils, perfluoro oils; volatile oils such as octamethylcyclotetrasiloxane, decamethylcyclopentasiloxane, hexademethylcyclohexasiloxane, heptamethylhexyltrisiloxane, heptamethyloctyltrisiloxane or C₈-C₁₆ isoparaffins, and isododecane.

21. Composition according to any one of the preceding claims, characterized in that the liquid fatty phase is present in a content ranging from 5% to 97% by weight and especially from 20% to 85% by weight relative to the total weight of the composition.

22. Composition according to any one of the preceding claims, characterized in that it also comprises at least one fatty substance that is pasty or solid at room temperature, chosen from waxes, gums and mixtures thereof.

23. Composition according to any one of the preceding claims, characterized in that it is in the form of a water-in-oil or oil-in-water emulsion.

24. Composition according to any one of claims 1 to 22, characterized in that it is anhydrous.

25. Composition according to any one of the preceding claims, characterized in that it also comprises at least one additional pulverulent compound chosen from fillers, pigments and nacres, and mixtures thereof.

26. Composition according to any one of the preceding claims, characterized in that it is in the form of a makeup composition.

27. Composition according to any one of the preceding claims, characterized in that it is in the form of a foundation, a concealer product, an eyeshadow, a makeup rouge, an eyeliner, an antisun composition, a skin-colouring composition or a body makeup composition.

28. Composition according to claim 27, characterized in that it is a foundation.

29. Cosmetic process for caring for and/or making up the skin and/or the lips, comprising at least one step of applying to the skin and/or the lips a composition according to any one of the preceding claims.

30. Use of silicate particles as defined according to any one of claims 2 to 12, in a matting cosmetic composition to obtain a long-lasting matt effect.

31. Use according to claim 30, wherein said silicate particles are associated with at least one film-forming polymer chosen from liposoluble, lipodispersible film-forming polymers, and blends thereof.
